(19) United States
(12) Patent Application Publication
(10) Pub. No.: US 2006/0111557 A1
(43) Pub. Date: May 25, 2006

(54) Water soluble toxin produced by pfiesteria species

(76) Inventors: JoAnn Burkholder, Raleigh, NC (US); Howard Glasgow, Bahama, NC (US); John S. Ramsdell, Charleston, SC (US); Peter D.R. Moeller, Charleston, SC (US); Stephan L. Morton, Charleston, SC (US)

Correspondence Address: MYERS BIGEL SIBLEY & SAJOVEC, PO BOX 37428, RALEIGH, NC, 27627, US

(21) Appl. No.: 10/530,151

(22) PCT Filed: Oct. 17, 2003
(86) PCT No.: PCT/US03/33637

§ 371 Date: Jan. 6, 2006

Related U.S. Application Data
(60) Provisional application No. 60/420,862, filed on Oct. 24, 2002.

(51) Int. Cl. C07K 14/195

(52) U.S. Cl. 530/350; 435/69.3; 435/252.3

(57) ABSTRACT

An isolated and purified Pfiesteria toxin having the NMR spectra given in FIG. 1 herein is described. The toxin may be produced by (a) culturing a Pfiesteria species in a growth media to produce Pfiesteria toxin therein; (b) separating a first fraction of organic compounds including said Pfiesteria toxin from said growth media; and then (c) separating a second fraction consisting essentially of said Pfiesteria toxin from said first fraction by chromatography with porous silica beads.

### FIELD OF THE INVENTION

The present invention relates to novel procedures for the isolation and purification of Pfiesteria toxin, as well as a description of the structural characterization of the toxin.

### BACKGROUND OF THE INVENTION

Pfiesteria toxin is a water-soluble molecule isolated from clonal cultures of the estuarine dinoflagellate pfiesteria piscida and Pfiesteria schumwayae. The toxin is responsible for fish kills and is implicated in human poisoning. While the deleterious effects of this toxin in humans have not been fully characterized, it has been reported that humans exposed to the toxin have suffered serious medical problems such as skin lesions, blindness, immune system repression, memory loss, and learning disabilities. See, e.g., A. Rezvani et al., Specificity of Cognitive Impairment from Pfiesteria piscicida exposure in rats, Neurotoxicology and Teratology 23, 609-616 (2001); E. Levin, A rat model of the cognitive impairment from Pfiesteria piscicida exposure, Environmental Health Perspectives 109, 757-763 (2001); A. Melo et al., Microfluorimetric analysis of a purinergic receptor (P2X₇) in GH₄C₁ Rat Pituitary Cells: Effects of a bioactive substance produced by Pfiesteria piscicida, Environmental Health Perspectives 109, 731-734 (2001); E. Fairley et al., Reporter gene assay for fish-killing activity produced by Pfiesteria piscicida, Environmental Health Perspectives 107, 711-714 (1999).

To date, the isolation and characterization of Pfisteria toxin has been difficult. See generally P. Moeller et al., Current Progress in Isolation and Characterization of Toxins Isolated from Pfiesteria piscida, Environmental Health Perspectives 109, 739-743 (2001).

### SUMMARY OF THE INVENTION

The present invention comprises isolated and purified Pfiesteria toxin, along with compositions comprising, consisting of, or consisting essentially of the same, having the NMR spectra given in FIG. 1 herein. By “consisting essentially of” is meant a composition containing Pfiesteria toxin sufficiently free of other organic compounds so that an NMR spectra as given in FIG. 1 herein can be produced from that composition.

Methods of isolating and purifying Pfiesteria toxin are also described herein.

The present invention is explained in greater detail in the drawings herein and the specification set forth below.

### BRIEF DESCRIPTION OF THE DRAWINGS

FIG. 1 shows a 13C NMR spectra of fractions containing compounds isolated from a Pfiesteria culture batch.

FIG. 2 shows a 13C NMR spectra of a fraction containing an identical compound as shown in FIG. 2, but isolated from a distinct Pfiesteria culture batch.

FIG. 3 shows an HPLC chromatrogram for the active fractions shown in FIGS. 1-2.

FIG. 4 shows the HPLC chromatograph for the most polar (far right) peak shown in the chromatogram of FIG. 3.

FIG. 5 shows a 13 NMR spectra of the most polar (far right) set of peaks shown in the chromatogram of FIG. 4.

### DETAILED DESCRIPTION OF THE PREFERRED EMBODIMENTS

The present inventors have identified and characterized the Pfiesteria toxin on the basis of its toxicity to GH4C1 rat pituitary cells (determined by MTT cytotoxicity, and/or c-fos luciferase reporter gene assay, and/or YO-PRO-1 uptake) and by its fish-killing activity (in the sheephead minnow species Cyprinodon variegatus). The toxin has been purified by normal phase chromatography utilizing beaded silica as the solid support. The active fraction has a unique NMR spectrum and exhibits a mass under 500 AMU.

Pfiesteria toxin as described herein can be produced by: (a) culturing a Pfiesteria species in a growth media to produce Pfiesteria toxin therein; (b) separating a first fraction of organic compounds including said Pfiesteria toxin from said growth media; (c) separating a second fraction consisting essentially of said Pfiesteria toxin from said first fraction by chromatography with porous silica beads. In a preferred embodiment, the porous silica beads are Iatrobeads. Any Pfiesteria species can be utilized as a source of the toxin described herein, including Pfiesteria piscicida and Pfiesteria shumwayae.

The isolated and characterized toxin of the present invention finds particular use as a standard for diagnostic tests for fish kill events, seafood safety, and human intoxication. In that the toxin is implicated in human neurological disorders, the toxin is also useful in the development of therapeutics for diseases of neurological, immunological or endocrine origin that involve dysfunction of purinergic P2X receptors and related ion-conducting pathways, and for the treatment of diseases involving dysfunction of purinergic P2X receptors in human or mammalian (e.g., dog, cat, rabbit, horse, cattle) subjects for medical or veterinary therapeutic purposes. The compound of the invention is useful as a ligand, when immobilized on a solid support, for binding and hence purifying P2X receptors, is useful when radiolabelled for identifying P2X receptors in tissue or tissue samples, is useful in vitro for activating or inhibiting P2X receptors, and is useful as a toxin per se when administered to fish species in a controlled manner for the eradication of a fish (e.g., when it is desired to remove or eradicate undesired or undesirable fish from a pond or the like).

The present invention is defined in greater detail in the following non-limiting Examples.

### EXAMPLES

I. General Protocol and Spectral Interpretation

This section provides an overview of the protocol for isolation and purification of Pfiesteria toxin, and the spectral characterization thereof. Specific protocol details are given further below.

Isolation and purification proceeds according to the following general steps:

Step 1: Trap organics from seawater cell growth medium on C18 column.

Step 2: Wash C18 with 2-3 void volumes of fresh water to elute excess salts

Step 3: Elute active metabolites (toxins) with Methanol and ethyl acetate, combine the organic elutants, dry and take residue onto an Iatrobead silica column.

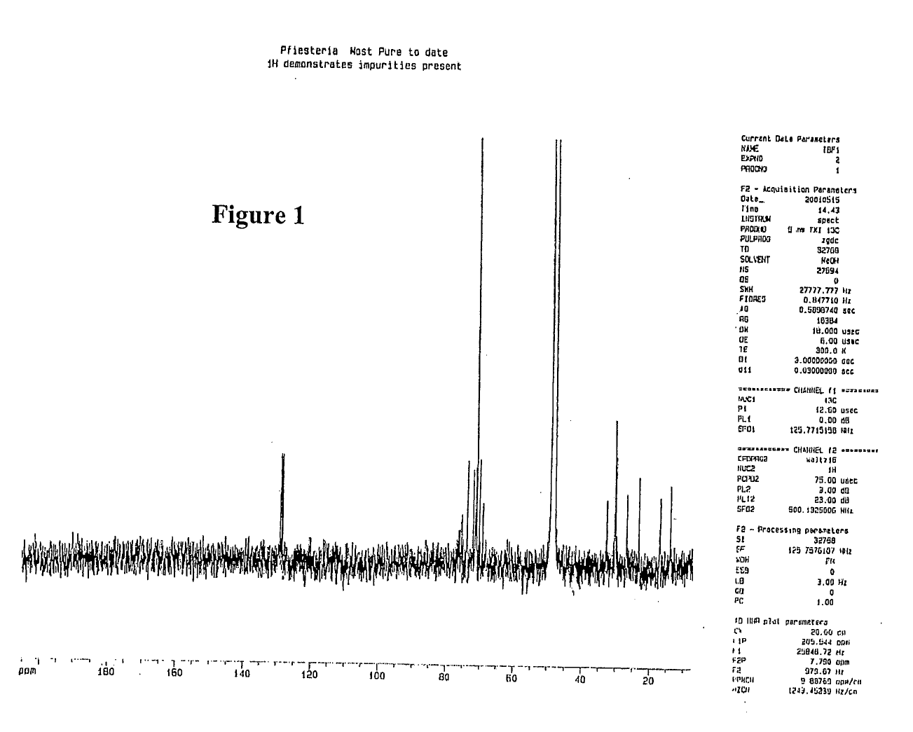

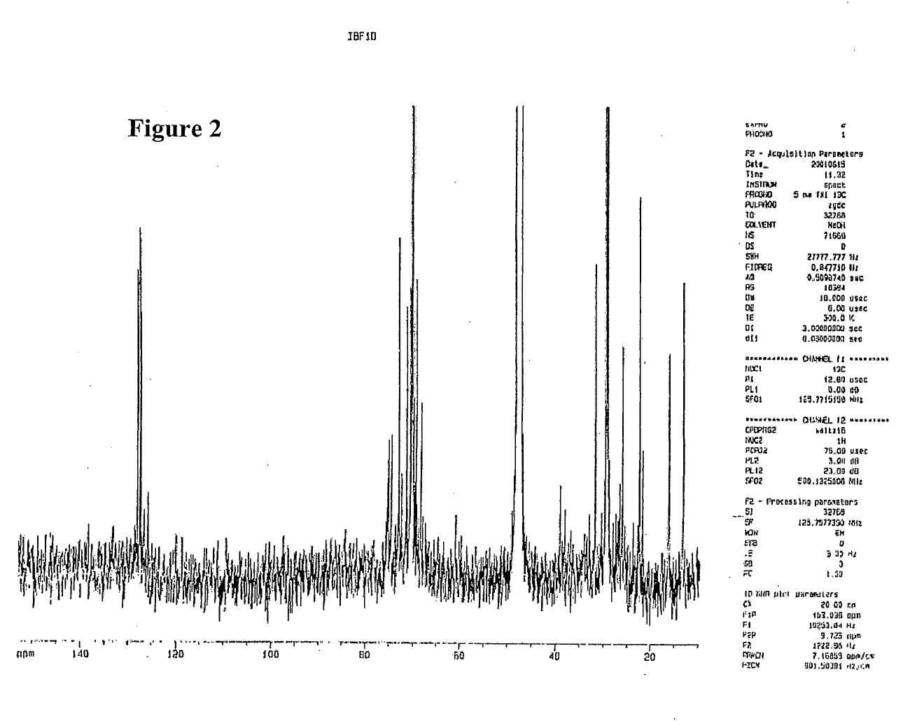

Step 4: The Iatrobead column is washed with high organics to elute non-polar contaminants and phthalic esters. This is followed by elution of active compounds with 1:1 MeoH:H2O. This elutant is again dried, NMR spectra taken and then solid residue taken on to HW40F size exclusion chromatography. Two 13C NMR spectra of fractions containing identical compounds isolated from distinct Pfiesteria culture batches as obtained from in this process are shown herein as FIGS. 1-2.

Step 5: The HW40F column is operated in an isocratic fashion with 20% Acetonitrile/water. The Active bands elute after copious salts and small proteinaceous materials (visible) typically in tubes 20-50. These fractions are combined, dried and the residue taken up in @ 50 microliters of water and diluted to two ml with acetonitrile. This solution is filtered through a 0.45 micron syringe filter and loaded onto the HPLC for normal phase chromatography using a polymeric amino (NH2P) column.

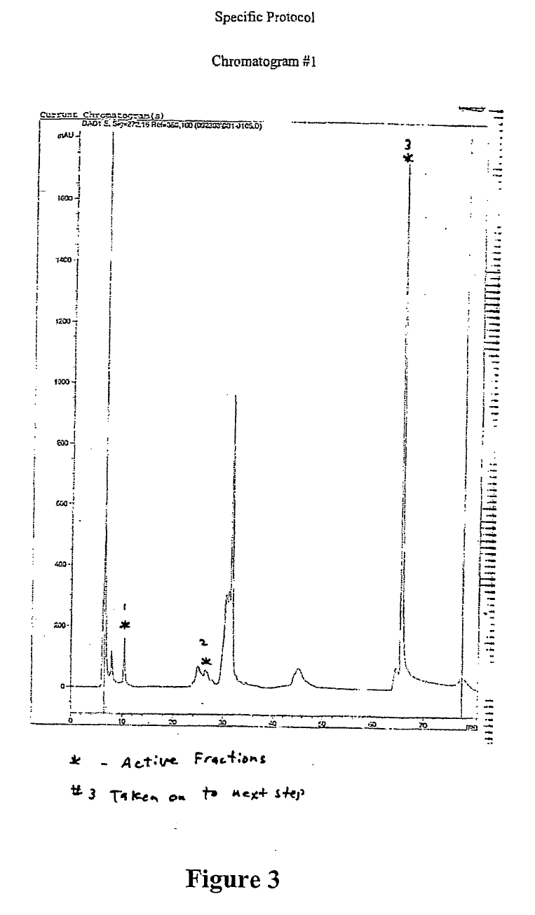

Step 6: The first amino column parameters are given below and the chromatogram given as FIG. 3, with marked peaks designating biologically active peaks (defined as both active in the GH3C1 cytotoxicity assay as well as able to kill sheepshead minnows in under 24 hours.)

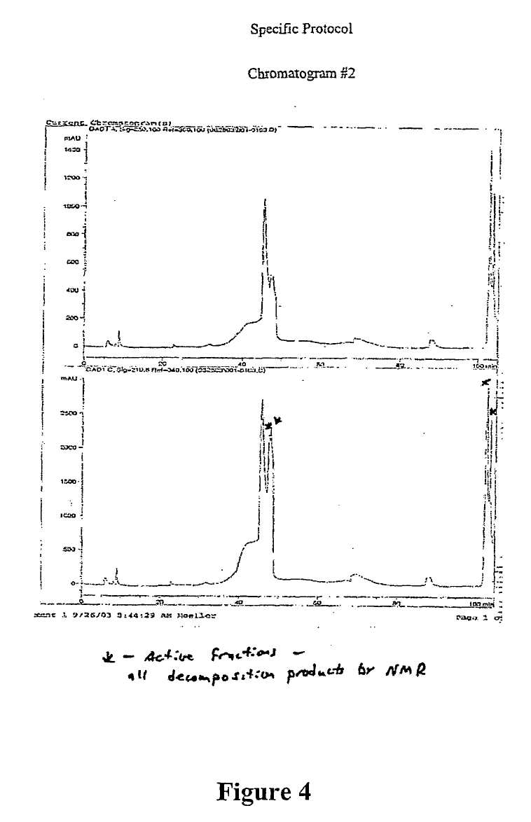

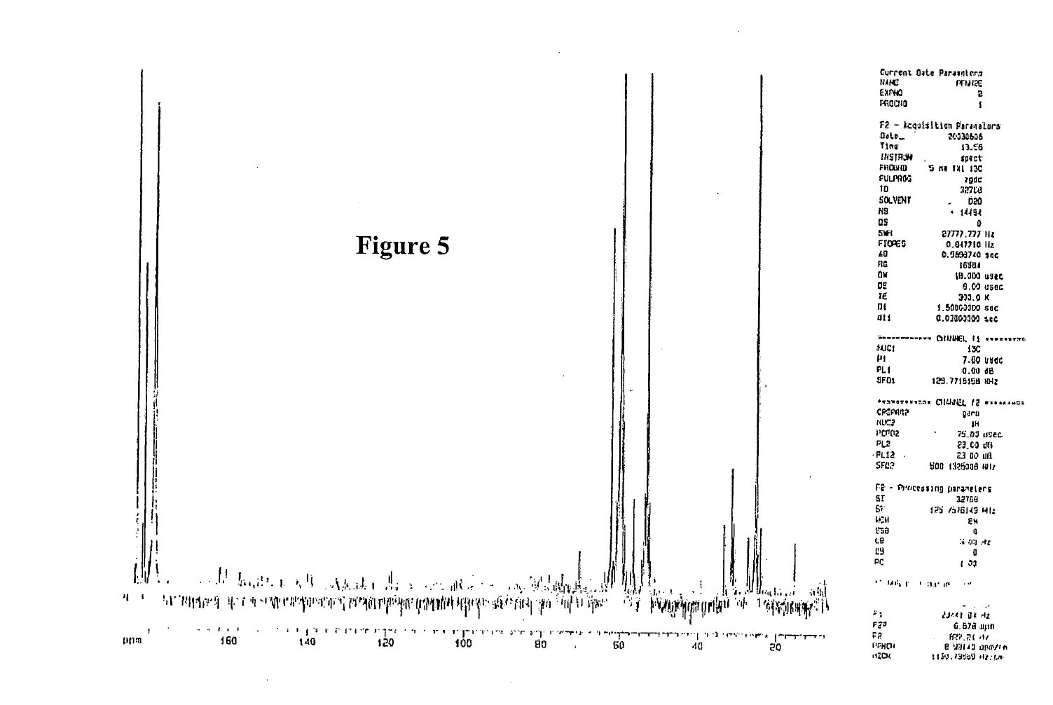

Step 7: The most polar (far right peak in the chromatogram generated in step 6 and shown as FIG. 3) was taken on to the next amino run using a different elution scheme (HPLC parameters are given below and the chromatogram given as FIG. 4). The active peak from Step 6 was split into 4 biologically active peaks in this subsequent HPLC experiment as marked on the chromatogram shown in FIG. 4. Activity was defined as above using cell cytotoxicity and fish bioassay. NMR data of the most active of these compounds (most polar or far right set of peaks) is shown as FIG. 5. This spectra is almost likely a breakdown or decomposition product of the parent molecule as defined in the NMR spectrum from Step 4, as only minor resonances are identifiable from previous NMR spectra. It appears that an oxidation step resulting in loss of carbon to carbon double bonds has resulted in carbon to oxygen double bonds in the form of a tricarboxylic acid.

NMR notes. The 13 C NMR spectrum from Step 4 (Iatrobead column) has been uniform over 3 years—i.e. the NMR spectrum of this active fraction (attached) is reproducible and consistent from culture batch to culture batch. This spectrum depicts a series of heteroatom bonded carbons (heteroatom can typically be: Nitrogen, Sulfur, Phosphorus, chlorine, Bromine, Iodine, Fluorine) as defined by the peaks from @60 ppm to 80 ppm. In addition the carbon-carbon double bonds defined by the peaks at @125-130 ppm are also characteristic peaks of this active fraction. The two very tall peaks cut off in the spectrum are solvent related. Those peaks to the right of 40 ppm are most certainly part of the main component of this fraction and are fingerprint carbons that are SP3 hybridized and bound only to hydrogen (this means each of these carbons have four bonds each and these bonds are only to other carbons or hydrogen.

These results indicate that the main component of this fraction has a polyhydroxylated or possibly a polyether-like structure with several double bonds. There may be a carbonyl (C doubly bonded to oxygen) to the left of 180 ppm but it is not clearly defined in the best spectrum. Molecular weight appears to be in the 400 to 500 atomic mass unit range as defined by the 13C NMR spectra. To date electrospray mass spectrometry (ESI-MS) in + and − mode, Atmospheric pressure chemical ionization MS (APCI-MS) and matrix assisted laser desorption MS (Maldi-MS) have failed to yield an interpretable molecular ion.

II. Protocol Details

This section provides further details for the steps described in general terms above.

A. Culture

1. The culture was grown in an environment chamber set at 25° C. with 35 μmol/photon/m²/sec with a 16:8 light/dark cycle. Pfiesteria cultures were grown in 36 L Kontes Cytostir flasks. The Pfiesteria cells are feed 6 L of Rhodomonas at approximately 1×10⁸ cells/L. the Rhodomonas sp. (CCMP767) food cultures were grown in 6 L Kontes Cytostir flasks enriched with f/2 media. Once stationary phase was reached in each Cytostir flask the batch was harvested using the methods listing in section B below.

B. Culture Harvest

1. The culture media is passed through a filter (Gelman Instrument Company model # 11873, filters are extra thick glass fiber 293 mm manufactured by Pall Corporation) to remove cell mass and physical debris. After the press, the culture media is pumped onto a column (14″ tall×5″- 13/16″ I.D. w/ ½″ medium coarse frit w/ another compression frit placed on top solid support) charged with 1 kg of C-18 (Sepra C-18 End-Capped 10 μm) which trapped the organics from the culture media.

C. Solvent/Solvent Scheme

1. After addition of all culture media, the column is flushed with 2 void volumes of de-ionized water to elute residual salts.

2. Elution of the toxin is carried out with 4 L of methanol+4 L of ethyl acetate to ensure complete recovery.

D. Toxin Purification/Chromatography

1. Once all the Pfiesteria culture is extracted in the methanol and ethyl acetate fractions, they are combined and rotary evaporated to dryness in a 1 liter round bottom flask. The temperature setting on the water bath is set at 45° C. When dry, 60 g of Iatrobeads 6RS-8060 is added to the flask along with 200 ml of chloroform and 100 ml of methanol and placed back on the rotary evaporated and dried.

2. The contents of the 1 liter round bottom (Iatrobeads+Pfiesteria) is added to a column manufactured by Southeastern Laboratory Apparatus, Inc. The column specifications are 18: long by 1: I.D. with a 500 ml round reservoir. At the bottom of the column is a medium pore frit with a stop cock. The column is pre-filled with Iatrobeads up to a level that is 4″ below the reservoir, the 60 g previously dried will fill the rest of the column.

3. Elution begins with 500 ml of ethyl acetate added to the column. This fraction is discarded because the activity of the toxin is retained in the subsequent two fractions. The next step is to add 500 ml of methanol to the column, collect the eluent and rotary evaporate to dryness. The same is done with a 50/50 mixture of methanol and water equaling 500 ml. The residue in the flasks are combined with 4-5 ml of methanol and then added to HW40F size exclusion resin (Tosoh Bioscience 45 μm).

E. HW40F

1. The sample is loaded onto a HW40F column, which the dimensions are the same as the Iatrabead column, and eluted with 1.5 L of 80/20 water-acetonitrile into a fraction collector collecting 8 ml samples. Typically, the toxic fractions are found in the tubes #20-50 and verified with the GH4C1 cytotoxicity assay.

2. Active fractions are combined, rotary evaporated to dryness and brought up in a minimal amount of water (0.5 ml) and then diluted to 2 ml with acetonitrile. This solution is syringe filtered through a 0.45 μm filter and applied to an Agilent 1100 Series HPLC with a photodiode array detector.

F. HPLC

1. The HPLC is equipped with a NH2P Polymeric Amino column (Shodex Asahipak) 4.6×250 column. The compound is purified using the following eluent scheme:

| Time (min.) | Solvent Scheme |
|---|---|
| 0-10 | 100% acetonitrile |
| 10-55 | Linear gradient 100% acetonitrile to 100% |
| | HPLC grade water |
| 56-70 | 75% of 0.1% NH4OH and 25% HPLC grade water |
| 71-80 | 100% HPLC grade water |

The system is run at a flow rate of 0.5 ml/min with sample injection of 100 ul. The compound of interest is monitored at 254,272 nm. * Chromatogram Included. Active peaks are marked on Chromatogram #1 (electronic HPLC data attached).

2. Active fractions are found at (min. 40-42; 102-104) these fractions are combined, rotary evaporated and brought up in minimal water (0.5 ml) and diluted to 2 ml with acetonitrile. This sample is subjected to the same amino column with a new eluent scheme:

| Time (min) | 0.1% NH4OH | Acetonitrile % | 0.1% TFA | Water |
|---|---|---|---|---|
| 0-10 | 0.0 | 100 | 0.0 | 0.0 |
| 11-35 | 0.0 | 90 | 0.0 | 10 |
| 36-60 | 0.0 | 80 | 0.0 | 20 |
| 61-80 | 0.0 | 70 | 0.0 | 30 |
| 81-86 | 0.0 | 0.0 | 0.0 | 100 |
| 87-94 | 0.0 | 0.0 | 100 | 0.0 |
| 95-104 | 100 | 0.0 | 0.0 | 0.0 |
| 105 | 0.0 | 100 | 0.0 | 0.0 |

The sample is typically injected at 100 ul and a flow rate of 0.5 ml/min. (Chromatogram #2 is attached with electronic HPLC data.)
Chromatography Reagents:

HPLC used to purify Pfiesteria toxin are Agilent 110 series pumps equipped with the 1100 series Degasser for online degassing, a quaternary pump allowing the use of four distinct solvents, an autosampler, column oven and a photodiode array detector (PDA).

The HPLC system is coupled to a Gilson FC 205 Fraction Collector operating at collection rate of 1 minute/tube/experiment. This is the equivalent to collecting 0.5 ml fractions over a given chromatographic experiment. All collections are based on 19 injections with the fractions overlayed from one injection to the other. This results in @ 8-9 mls collected per tube prior to testing.

Iatrobeads are beaded silica and operated in the normal phase (that is less polar compounds elute first followed by more polar compounds).

Iatrobeads

6RS-8060

Lot 4016101

Shell-USA Inc.

Fredericksburg, Va. 22407

540-548-8010

C-18 trapping utilizes bulk C-18 described below.

C-18

Sepra C-18 End-Capped 04K-4348

Batch #=S201-20

1,000 g

Phenomenex

The foregoing is illustrative of the present invention, and is not to be construed as limiting thereof. The invention is defined by the following claims, with equivalents of the claims to be included therein.

That which is claimed is:

1. Isolated and purified Pfiesteria toxin having the NMR spectra given in FIG. 1 herein.

2. Isolated and purified Pfiesteria toxin according to claim 1 produced by the process of:
(a) culturing a Pfiesteria species in a growth media to produce Pfiesteria toxin therein;
(b) separating a first fraction of organic compounds including said Pfiesteria toxin from said growth media;
(c) separating a second fraction consisting essentially of said Pfiesteria toxin from said first fraction by chromatography with porous silica beads.

3. Isolated and purified Pfiesteria toxin according to claim 1, wherein said porous silica beads are Iatrobeads.

4. Isolated and purified Pfiesteria toxin according to claim 1, wherein said porous silica beads are 6RS-8060 Iatrobeads.

5. Isolated and purified Pfiesteria toxin according to claim 1, wherein said Pfiesteria species is selected from the group consisting of Pfiesteria piscicida and Pfiesteria shumwayae.

6. Isolated and purified Pfiesteria toxin according to claim 1, wherein said Pfiesteria species is Pfiesteria piscicidae.

7. Isolated and purified Pfiesteria toxin according to claim 1, wherein said Pfiesteria species is Pfiesteria shumwayae.

8. A method of isolated and purified Pfiesteria toxin, comprising the steps of:
(a) culturing a Pfiesteria species in a growth media to produce Pfiesteria toxin therein;
(b) separating a first fraction of organic compounds including said Pfiesteria toxin from said growth media;
(c) separating a second fraction consisting essentially of said Pfiesteria toxin from said first fraction by chromatography with porous silica beads.

8. The method of claim 7, wherein said second fraction is characterized by an NMR spectrum as given in FIG. 1 herein.

9. The method of claim 7, wherein said porous silica beads are Iatrobeads.

10. The method of claim 7, wherein said porous silica beads are 6RS-8060 Iatrobeads.

11. The method of claim 7, wherein said Pfiesteria species is selected from the group consisting of Pfiesteria piscicida and Pfiesteria shumwayae.

12. The method of claim 7, wherein said Pfiesteria species is Pfiesteria piscicidae.

13. The method of claim 7, wherein said Pfiesteria species is Pfiesteria shumwayae.
